SEQUENCE LISTING (10 sequences)

SEQ ID NO: 1 - DNA, 20 bp, Artificial sequence
  Synthetic primer
cgccaactatgaccaggata

SEQ ID NO: 2 - DNA, 18 bp, Artificial sequence
  Synthetic primer
gttgcccccagttacagt

SEQ ID NO: 3 - DNA, 24 bp, Artificial sequence
  Synthetic primer
atgggtcagaaggattcctatgtg

SEQ ID NO: 4 - DNA, 24 bp, Artificial sequence
  Synthetic primer
cttcatgaggtagtcagtcaggtc

SEQ ID NO: 5 - DNA, 18 bp, Artificial sequence
  Synthetic primer
tctcaaggaggtcggtat

SEQ ID NO: 6 - DNA, 18 bp, Artificial sequence
  Synthetic primer
tcggggcaataagaaagg

SEQ ID NO: 7 - DNA, 21 bp, Artificial sequence
  Synthetic primer
aatgccacaggtttccggttc

SEQ ID NO: 8 - DNA, 21 bp, Artificial sequence
  Synthetic primer
acaccccacagccagcactat

SEQ ID NO: 9 - DNA, 24 bp, Artificial sequence
  Synthetic primer
atgggtcagaaggattcctatgtg

SEQ ID NO: 10 - DNA, 24 bp, Artificial sequence
  Synthetic primer
cttcatgaggtagtcagtcaggtc

(19) United States
(12) Patent Application Publication
(10) Pub. No.: US 2009/0082322 A1
(43) Pub. Date: Mar. 26, 2009

(54) Gender Differences in Experimental Aortic Aneurysm Formation

(75) Inventor: Gilbert Upchurch, JR., Ann Arbor, MI (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: THE REGENTS OF THE UNIVERSITY OF MICHIGAN, Ann Arbor, MI (US)

(21) Appl. No.: 11/664,944

(22) PCT Filed: Oct. 11, 2005
(86) PCT No.: PCT/US2005/036282

§ 371 Date: Aug. 18, 2008

Related U.S. Application Data
(60) Provisional application No. 60/617,623, filed on Oct. 9, 2004.

(51) Int. Cl. A61K 31/566; A61P 9/00

(52) U.S. Cl. 514/182; 514/169

(57) ABSTRACT

The present invention generally relates to the relevance of gender differences on abdominal aortic aneurysm (AAA) formation and to methods of inhibiting, preventing, and/or treating AAA formation by administering estrogen, and estrogen derivative, and/or estrogen receptor agonist, to an organism in need thereof.

### GOVERNMENT INTERESTS

The U.S. Government owns rights in the invention pursuant to National Institute of Health grant number K08 (HL67885-02).

### BACKGROUND OF THE INVENTION

Abdominal aortic aneurysms (AAAs) are potentially life-threatening, accounting for 150,000 hospital admissions yearly. Clear gender differences exist, with a prevalence in men 4-times that in women. (Singh K, et al., Am J Epidemiol., 154:236-244 (2001); Pleumeekers H J, et al., Am J Epidemiol., 142:1291-1299 (1995)). The diminished risk of AAA development is lost in women after menopause, suggesting that reproductive events, including circulating estrogens, play a protective role. (La Vecchia C, et al., Am J Obstet Gynecol., 157:1108-1112 (1987); Bengtsson H, et al., In: Tilson M D, Boyd C D, eds. The Abdominal Aortic Aneurysm: Genetics, Pathophlysiology, and Molecular Biology, 1-24 (1996)).

Prominent local inflammatory cell infiltration, aortic wall cytokine production, medial wall destruction by proteinases, and smooth muscle cell depletion characterize most AAAs. Destruction of elastin and collagen in the media by various matrix metalloproteinases (MMPs) is considered an essential element of AAA formation. (Freestone T, et al., Arterioscler Thromb Vasc Biol., 15:1145-1151 (1995); Tamarina N A, et al., Sugery, 122:264271 (1997); Thompson R W, Parks W C., Ann NY Acad Sci., 800:157-174 (1996)). MMP-1, MMP-2, MMP-3, MMP-9, MMP-12, as well as tissue inhibitor of metalloproteinase-1, are all upregulated in the walls of human AAAs. (Allaire E, et al., J Clin Invest., 102:1413-1420 (1998); Pyo R, et al., J Clin Invest., 105:1641-1649 (2000); Curci J A, et al., J Clin Invest., 102:1900-1910 (1998); Carrell T W, et al., Circulation, 105:477-482 (2002); Davis V, et al., Arterioscler Thromb Vasc Biol., 18:1625-1633 (1998); Thompson R W, et al., J Clin Invest., 96:318-326 (1995)). Two of these, MMP-2 and MM-9, have been extensively studied. MMP-9 has attracted particular attention in that it is highly expressed in human AAA wall and is present in serum from AAA patients. (McMillan W D, J Vasc Surg., 29:122-127 (1999); Thompson R W, et al., J Clin Invest., 96:318-326 (1995)). Mice with deletion of the gene responsible for the MMP-9 protein are resistant to the development of experimental AAAs. (Pyo R, et al., J Clin Invest., 105:1641-1649 (2000)). In addition, MMP-2, derived from aortic mesenchymal cells, appears necessary for experimental aneurysm formation. (Longo G M, et al., J Clin Invest., 110:625-632 (2002)).

Many studies implicating MMPs in AAA evolution have used a rat or mouse model with porcine pancreatic elastase perfusion of the infrarenal aorta. This model causes an initial influx of macrophages and lymphocytes leading to destruction and remodeling of the aortic wall matrix, and subsequent aneurysm development. (Anidjar S, et al., Circulation, 82:973-981 (1990)) Atherosclerosis, once considered essential to aneurysm development, is not thought to be the mechanism responsible for AAA formation. (Shteinberg D, et al., Eur J Vasc Endovasc Surg., 20:462-465 (2000); Agmon Y, et al., J Am Coll Cardiol., 42:1076-1083 (2003)) Importantly, nearly all previous studies have been performed using male rodents. To date, the influence of gender on experimental AAA formation has received little attention. Furthermore, although estrogen is known to affect collagen and elastin matrix remodeling in rats (Fischer G M, Swain M L., Exper Mol Pathol., 33:15-24 (1980)), its role in AAA formation has not been studied.

### SUMMARY OF THE INVENTION

This investigation was designed to determine the relevance of male and female gender on experimental AAA formation and to define local and systemic events that might influence any anticipated differences related to gender.

Accordingly, one embodiment of the present invention provides a method of inhibiting abdominal aortic aneurysm (AAA) formation comprising administering a therapeutically effective amount of a compound selected from the group consisting of estrogen, an estrogen derivative, and an estrogen receptor agonist, to an organism in need thereof. In another embodiment, a method of preventing AAA formation comprising administering a therapeutically effective amount of a compound selected from the group consisting of estrogen, an estrogen derivative, and an estrogen receptor agonist, to an organism in need thereof is provided.

In still another embodiment of the invention, a method of inhibiting aortic macrophage infiltration is provided comprising administering a therapeutically effective amount of a compound selected from the group consisting of estrogen, an estrogen derivative, and an estrogen receptor agonist, to an organism in need thereof.

In yet another embodiment, a method of inhibiting macrophage-derived matrix metalloproteinase (MMP) production comprising administering a therapeutically effective amount of a compound selected from the group consisting of estrogen, an estrogen derivative, and an estrogen receptor agonist, to an organism in need thereof is provided. In a related aspect, the matrix metalloproteinase is selected from the group consisting of MMP-1, MMP-2, MMP-3, MMP-9, MMP-12, and combinations thereof. Sequences of the aforementioned matrix metalloproteinases are well known in the art and readily available in public databases (e.g., GenBank).

Another embodiment of the invention provides a method of improving the healing of an AAA repair surgery in an organism, comprising administering a therapeutically effective amount of a compound selected from the group consisting of estrogen, an estrogen derivative, and an estrogen receptor agonist, to an organism in need thereof. In yet another embodiment, a method of reducing the size of one or more AAAs is provided comprising administering a therapeutically effective amount of a compound selected from the group consisting of estrogen, an estrogen derivative, and an estrogen receptor agonist, to an organism in need thereof.

In a related embodiment, the aforementioned derivative is estradiol. In another related embodiment, the aforementioned organism in need thereof is a mammal. In a preferred embodiment, the mammal is a human. In yet another preferred embodiment, the human is an adult.

### DETAILED DESCRIPTION

Generally, the invention involves a method of treating, inhibiting, reducing the risk of, and/or preventing abdominal aortic aneurysm (AAA) comprising administering an agent at a therapeutically effective dosage in an effective dosage form at a selected interval. Treating, inhibiting, reducing the risk of or preventing AAA refers to any observable beneficial effect of the treatment, including modulation of underlying biological pathways which participate in AAA, as well as modulating a biological response associated with an AAA-related pathway. In one aspect then, methods of the invention inhibit aortic macrophage infiltration, and in another aspect, methods of the invention inhibit macrophage-derived matrix metalloproteinase (MMP) production. The beneficial effect can be evidenced by a delayed onset or progression of AAA, a reduction in the severity of some or all of the clinical symptoms, or an improvement in the overall health.

Methods of the invention contemplate use of an agent selected from estrogen, an estrogen derivative and an estrogen receptor agonist. Accordingly, any compound which produces an estrogen biological effect is amendable for use in the invention. Non-limiting examples of suitable estrogen and estrogen analogs and estrogen agonists are well known in the art and include estradiol, phytoestrogens, ethnyl estradiol, mestranol, 17 β-estradiol, 17 α-estradiol, tamoxifen derivatives such as 4-hydroxytamoxifer, and estriol (estra-1,3,5(10) -triene-3,16,17-triol, E3, such as estriol succinate, estriol dihexanoate or estriol sulfamate. Use of precursors, prodrugs or analogs of estriol (such as nyestriol), estrone or precursor or analogs of estrone, 17 β-estradiol (E2) or precursors (including aromatizable testosterone) or analogs of estradiol are also contemplated. In addition, metabolites and derivatives are further contemplated which may have a similar core structure to estrogen but may have one or more different groups (ex. hydroxyl, ketone, halide, etc.) at one or more ring positions. Useful agents may also be an agonist of either estrogen receptor α or β or both.

Suitable estrogens and analogs and estrogen antagonists may be isolated from natural sources, or synthesized by chemical or recombinant methods. Many recombinant and synthetic estrogens and estrogen agonists are commercially available and are the subject of numerous issued patents. Synthetic steroids which are effective on estrogen receptor are also useful in methods of the invention, such compounds include those described in WO 97/08188 or U.S. Pat. No. 6,043,236, the disclosure of which are incorporated herein by reference in their entireties.

Compounds useful in the methods may be steroidal or non-steroidal in nature. The art is replete with estrogen-like compounds and estrogen receptor agonists which are useful in methods of the invention. See e.g., U.S. Pat. Nos. 5,843,934, 6,358,943, 6,355,670, 6,355,623, 6,355,630, 6,352,970, 6,331,562, 6,326,366, 6,323,190, 6,316,494, 6,274,618 each of which is incorporated by reference in its entirety. See also, useful estrogenic compounds as disclosed in United States Patent Application 20040198670 and United States Patent Application 20040110824, the disclosures of which are incorporated herein in their entireties. Still further, estrogen receptor modulators contemplated for use in the invention are exemplified by those disclosed in U.S. Patent Application 20040162304, U.S. Patent Application 20040167112, U.S. Patent Application 20040127576; U.S. Patent Application 20040110767; U.S. Patent Application 20040102498; U.S. Patent Application 20040082575; U.S. Patent Application 20040077701; and U.S. Patent Application 20040039015, the disclosures of which are incorporated by reference in their entireties.

Any one or combination of these estrogens or estrogen receptor active agents may be used in methods of the invention. The selection of the estrogens or estrogen receptor active agents can be made considering, for example, secondary side effects of the treatment to the patient and the individual patient's response to a first treatment regimen. Accordingly, any treatment regimen may be modified during the course of treatment as deemed necessary by an attending physician.

A therapeutically effective dose of an agent is one sufficient to raise the serum concentration above basal levels and achieve the desired benefit. In general, the dosage will depend on the particular estrogen compound, and the particular patient. Typically, the dosage of estrogen active agent will range from about 0.001 to 200 mg/day, more typically from about 0.01 to 20 mg/day and most typically from about 0.1 to 10 mg/day, the dosage being dependent in one aspect on the achievement of a predetermined circulating level of the estrogen active agent. By way of example only, in one embodiment, where the agent is estriol, the preferable oral dose is from about 4 to 16 milligrams daily, or in the alternative, about 8 milligrams daily. In this embodiment, blood serum levels reach at least about 2 ng/ml, may reach about 10 to about 35 ng/ml, or about 20-30 ng/ml. In another exemplary embodiment, estradiol levels would reach at least about 2 ng/ml to about 10-35 ng/ml. In other exemplary embodiments, estrone levels would reach at least about 2 ng/ml to about 5-18 ng/ml. The worker of ordinary skill in the art will readily appreciate however, that the dosage of the agent may be selected for an individual patient depending upon the route of administration, overall condition of the patient, age and weight of the patient, other medications the patient is taking and other factors normally considered by the attending physician, when determining the individual regimen and dosage level as the most appropriate for a particular patient. As estrogen therapy can sometimes cause adverse side-effects the subject will be monitored for signs of any increased urine excretion, weight gain, changes in breast or uterine tissues will be monitored. If such side effects are observed, the particular estrogen active compound and/or the treatment regimen may be modified accordingly.

The therapeutically effective dose of an agent included in the dosage form is selected at least by considering the type of agent selected and the mode of administration. The dosage form may include the active agent in combination with other inert ingredients, including adjutants and pharmaceutically acceptable carriers for the facilitation of dosage to the patient as known to those skilled in the pharmaceutical arts. The dosage form may be any form suitable to cause the agent to enter into the target tissue of the patient.

In one embodiment, the dosage form of the agent is an oral preparation (liquid, tablet, capsule, caplet or the like) which when consumed results in elevated serum estrogen activity levels. The oral preparation may comprise conventional carriers including diluents, binders, time release agents, lubricants and disintegrants.

In other embodiments, the dosage form of the agent may be provided in a topical preparation (lotion, cream ointment or the like) for transdermal application to the extent that the transdermal administration permits delivery of the agent to the desired tissue. See, e.g., U.S. Pat. No. 4,906,475, the disclosure of which is incorporated herein in its entirety. Alternatively, the dosage form may be provided in a suppository or the like for transvaginal or transrectal application.

In still additional embodiments, the dosage form may also allow for preparations to be applied subcutaneously, intravenously, intramuscularly, intranasal or via the respiratory system (i.e., inhalation). For administration by injection, it is preferred to use the compound in solution in a sterile aqueous vehicle which may also contain other solutes such as buffers or preservatives as well as sufficient quantities of pharmaceutically acceptable salts or of glucose to make the solution isotonic. Subcutaneous injection is the preferred route of administration. Dosages are essentially the same as those set forth above for oral administration. Formulations for each of these routes of administration are well known in the art as described in U.S. Patent Application 20050152896, the disclosure of which is incorporated herein in its entirety.

Any one or a combination of active agents as described herein and otherwise known in the art may be included in the dosage form with the primary agent. Alternatively, any one or a combination of active agents may be administered independently of each other, but concurrent in time such that the patient is exposed to at least two agents intended to achieve the desired beneficial result.

In another aspect, secondary agents be administered, either at the same time that one or more estrogen-active agents are administered or within a time frame such that the secondary agent acts synergistically with the one or more estrogen-active agents. Secondary agents may be selected to enhance the effect of the estrogen or estrogen receptor active agent or effect a different system than that effected by the estrogen or estrogen receptor active agent.

The following examples present preferred embodiments and techniques, but are not intended to limit the scope of the invention. Those of skill in the art will, in light of the present disclosure, appreciate that many changes can be made in the specific materials and methods which are disclosed and still obtain a like or similar result without departing from the spirit and scope of the invention. In the following examples, Example 1 discloses gender differences in AAA formation, Example 2 discloses the epidemiology of surgically treated AAAs in the United States during the time period 1988 to 2000, Example 3 discloses differential effect of 17-β-estradiol on smooth muscle cell and aortic explant MMP-2, and Example 4 discloses gender differences in rat aortic smooth muscle cell matrix MMP-9.

### EXAMPLE 1

### Gender Differences in Experimental Aortic Aneurysm Formation

### I. Materials and Methods

A. Animals

Sprague-Dawley rats (200 to 250 grams, age 8 to 10 weeks), obtained from Charles River Laboratories (Wilmington, Mass.), were used in all experiments. Procedures and experiments were approved by the University of Michigan Universal Committee on the Use and Care of Animals (#8220 and #8314).

B. Elastase Perfusion Aneurysm Model

Pancreatic porcine elastase perfusion of the rat aortas was performed as described previously. (Anidjar S, et al., Circulation, 82:973-981 (1990)). Male and female rats (n=15, each) were anesthetized with 2-2.5% isoflurane inhalation, and the infrarenal abdominal aorta was isolated under sterile conditions. Digital video micrometry was performed to directly measure outer aortic diameter. Specifically, images of the aorta were obtained using a Spot Insight Color Optical Camera (Diagnostic Instruments, Sterling Heights, Mich.) attached to an operating microscope (Nikon, Melville, N.Y.). Aortic diameters were then measured at the level of the left renal vein, the mid-infrarenal aorta, and the aortic bifurcation in triplicate using Image Pro Express software (Media Cybernetics, Inc, Silver Spring, Md.). Temporary proximal and distal aortic control was obtained using temporary 4-0 cotton suture loops, following which an aortotomy was made near the aortic bifurcation with a 30-gauge needle. The infrarenal aorta was cannulated with PE-10 tubing and perfused with 12 U of porcine pancreatic elastase diluted to a total volume of 2 mL with sterile normal saline (Lot #032K7660 or Lot #102K685; Sigma, St. Louis, Mo.) over 60 minutes. Subsequently, the tubing was removed and the aortotomy repaired with 10-0 monofilament suture. Patency was assured in all cases. Aortic diameter measurements were repeated immediately after perfusion. The intestines were replaced; the abdominal wall was closed; and the rats were recovered. At 7 or 14 days, aortas were re-exposed and aortic diameters were re-measured in vivo. Aneurysm formation was defined as a 100% increase in an individual animal's pre-elastase perfusion aortic diameter. The infrarenal aorta was then removed and subjected to histological study, immunohistochemistry, and quantitative polymerase chain reaction (PCR).

C. Aortic Transplantation

In additional rats, transplantation of the infrarenal rat aorta was performed as previously described. (Ailawadi G, et al., J Vasc Surg., 37:1059-1066 (2003)). Briefly, male and female donor rats were anesthetized and the abdominal aortas isolated. Donor rats were anticoagulated with 300 U of heparin and the abdominal aorta was rapidly removed and placed in cold 0.9% normal saline. The recipient rats' infrarenal abdominal aortas were similarly isolated and proximal and distal aortic control was obtained with temporary 4-0 cotton suture loops. The recipient abdominal aorta was excised and donor abdominal aorta was transplanted into the infrarenal position of a size-matched recipient using a running 10-0 monofilament suture in an end-to-end fashion. After aortic patency was assured, the abdominal incision was closed and rats were recovered.

Female donor aortas were transplanted into male recipients (n=7) with controls including female aortas transplanted into female recipients (n=7) and male aortas transplanted into male recipients (n=9). Fourteen days after transplantation, the transplanted aortas were subjected to pancreatic porcine elastase perfusion and harvested after 14 days as described herein.

D. Estrogen Pellet Implantation

In other experiments, male rats were randomized to implantation of an estrogen pellet or sham implantation (n=13, each). The former involved the subcutaneous implantation of a 21-day slow-release 0.1-mg 17-β-estradiol pellet (Innovative Research of America, Sarasota, Fla.) in the posterior neck. Sham rats underwent the same implantation procedure without insertion of any pellet. This particular estradiol dose results in serum estradiol levels at 2 to 3 weeks of 44.7±6.1 pg/mL compared with 15.0±2.2 pg/mL in control rats (P<0.05). Rat aortas from both groups were subjected to elastase perfusion 5 days later. The aortas were then removed for study 7 or 14 days after elastase perfusion.

E. Histological Analysis

All excised aortas were fixed in 10% formalin for 18 hours, followed by immersion in 70% ethanol for 24 hours. Aortas were then imbedded in paraffin and 4-μm sections were prepared with hematoxylin and eosin and Verhoeff-Van Gieson stains.

Immunohistochemistry was undertaken after deparaffinization, rehydration, and unmasking using Trilogy (Cell Marque Corp, Hot Springs, Ariz.) in a Princess model pressure cooker (Cell Marque). Endogenous peroxidase activity was then blocked using 3% hydrogen peroxide in methanol. To help ensure that rejection was not occurring in elastase-perfused or transplanted animals, anti-T lymphocyte immunohistochemistry was performed. Specifically, antirat CD3 monoclonal antibody (BD Pharmingen, San Diego, Calif.) was used as the primary antibody and mouse IgG Vectastain (Vector Laboratories, Burlingame, Calif.) as the secondary antibody. Rat spleen was used as the positive control for anti-CD3 staining. ED-1 macrophage staining was performed using mouse antirat ED-1 primary antibody (Serotec, Raleigh, N.C.) and mouse IgG Vectastain secondary antibody (Vector Laboratories). MMP-9 immunohistochemistry was performed using rabbit antirat MMP-9 polyclonal primary antibody (Chemicon International, Temecula, Calif.) and rabbit IgG Vectastain secondary antibody (Vector Laboratories). Staining for all these antibodies was performed using Vector Red alkaline phosphatase (Vector Laboratories) followed by hematoxylin QS counterstain (Vector Laboratories).

Colocalization studies involved deparaffinization, rehydration, and unmasking as previously described. Endogenous peroxidase activity was blocked using 3% hydrogen peroxide in methanol. Staining for ED-1 was performed using mouse antirat ED-1 primary antibody (Serotec), mouse IgG Vectastain secondary antibody (Vector Laboratories), and Vector Blue alkaline phosphatase. Samples were stored in PBS at 4° C. overnight. The next day, staining for MMP-9 was performed using rabbit antirat MMP-9 polyclonal primary antibody AB19016 (Chemicon International), rabbit IgG Vectastain secondary antibody (Vector Laboratories), and Vector Red alkaline phosphatase stain.

F. Quantitative PCR

Expression of MMP-9 and β-actin mRNA was determined using quantitative PCR. Messenger RNA was isolated by exposure of aortas to TRIzol reagent and reverse-transcribed by incubating with oligo-dT primer (Life Technologies, Grand Island, N.Y.) and M-MLV Reverse Transcriptase (Life Technologies, Grand Island, N.Y.) at 94° C. for 3 minutes, followed by 40° C. for 70 minutes. The resultant cDNA was amplified by Taq Polymerase (Promega, Madison, Wis.) in a SmartCycler quantitative PCR system (Cepheid, Sunnyvale, Calif.). SYBR intercalating dye (Roche, Indianapolis, Ind.) was used to monitor cDNA amplification for each gene. MMP-9 and β-actin primer sequences were derived using Primer Premeir software (PREMIER Biosoft International, Palo Alto, Calif.) based on primary cDNA sequences from GenBank. Primer sequences are as follows:

MMP-9 forward primer, CGC CAA CTA TGA CCA GGA TA (SEQ ID NO: 1); MMP-9 reverse primer, GTT GCC CCC AGT TAC AGT (SEQ ID NO: 2); β-actin forward primer, ATG GGT CAG AAG GAT TCC TAT GTG (SEQ ID NO: 3); β-actin reverse primer, CTT CAT GAG GTA GTC AGT CAG GTC (SEQ ID NO: 4). Results were normalized using β-actin to account for variation in mRNA amounts. Quantification of mRNA levels used ΔCₜ values, calculated by the formula:

ΔCₜ=C_{ttargetgene}−C_{tβ-actin}.

Expression of the target gene in ratio to β-actin expression was calculated by the formula:

target gene expression/β-actin expression=2^{-(ΔCt)}

G. Substrate Gel Zymography

MMP-9 distribution after elastase perfusion was determined by zymography as previously described. (Eagleton M J, et al., J Surg Res., 104:15-21 (2002)). Gelatinase activity was evident by clear bands against a dark blue background. The molecular weight of each band was determined by comparison of the bands against samples containing human recombinant MMP-9 (Oncogene, Boston, Mass.). In previous studies, these bands were inhibited by EDTA and are thus metalloproteinases. (Eagleton M J, et al., J Surg Res., 104:15-21 (2002) supra). Semiquantitative measurements were performed using densitometry as described and normalized to total protein.

H. Densitometry

Gels were imaged with a FOTO/Analyst charge-coupled device CAMERA (Fotodyne, Hartland, Wis.). Band strengths were quantified using GEL-Pro Analyzer software version 3.1 (Media Cybernetics, Silver Springs, Md.).

I. Total Protein Assay

Total cellular protein was determined by a bicinchoninic acid protein assay (Pierce, Rockford, Ill.) in aortas on which MMP-9 activity assays were performed after they had been solubilized in 0.1% sodium dodecyl sulfate.

J. Data Analysis

Data are represented as mean±SE. Data were assessed by nonpaired t test or ANOVA with statistical significance assigned as P<0.05. When significance was reached, post hoc Tukey test was used to compare individual groups. Statistical analysis was performed using Prism software (GraphPad Software, San Diego, Calif.).

### II. Results

A. Baseline Histology in Male Versus Female Rats Male and female rat aortas not subjected to intervention were harvested and subjected to histological analysis. Male and female aortas were nearly identical in wall thickness and aortic lamellar structure by Verhoeff-Van Gieson stain. Aortas from both genders were indistinguishable by an experienced pathologist. CD3 immunohistochemistry demonstrated little to no lymphocytic infiltrate in the aortas of either males or females.

B. Experimental AAA Formation in Male Versus Female Rats

Preperfusion baseline aortic diameters were not different (P±0.20) between male and female aortas (1.41±0.16 versus 1.32±0.07, respectively). The mean increase in aortic diameter 14 days after elastase perfusion in male aortas was 200±37.6%, whereas female aortas had a mean aortic diameter increase of 69.4±26.5% (P=0.0234). The incidence of AAAs defined as an increase in aortic diameter at least 100% from preperfusion diameter was 82% in male rats compared with 29% in female rats (P=0.0229).

Male aneurysmal aortas exhibited circumferential disruption of the elastic lamellae 14 days after elastase perfusion, whereas the aortas of female rats after elastase perfusion had largely intact elastin fibers. CD3 staining demonstrated minimal lymphocyte infiltration in either male or female aortas. ED-1-positive cells were located primarily in the adventitia and media, consistent with previous reports. (Pyo R, et al., J Clin Invest., 105:1641-1649 (2000)). Macrophage infiltration was more prominent in the male aortas, where ED-1-positive cell counts were 6.2±1.0 cells/HPF compared with 0.541±0.02 cells/HPF in female aortas (P=0.003).

MMP-9 staining was also more evident in the media and adventitia of male aortas compared with female aortas, as evidenced by immunohistochemistry. Colocalization of ED-1 and MMP-9 demonstrated increased costaining of macrophages and MMP-9 in male aortas versus female aortas. Similarly, male aortas exhibited increased MMP-9 expression by quantitative PCR compared with female aortas (males, 0.39±0.09 versus 0.003±0.001 MMP-9 mRNA copies; P=0.001). Total MMP-9 activity by zymography was 369% greater in male than female aortas (P=0.022).

C. Aneurysm-Resistant Phenotype Is Lost After Transplantation of the Female Into the Male

All male and female aortas, when transplanted into male recipients and subsequently subjected to elastase perfusion, developed AAAs at 14 days, whereas only 17% of the female aortas transplanted into female recipients developed AAAs. Aortic dilations were similar among male-to-male and female-to-male transplanted aortas, but were significantly lower in the female-to-female transplanted aortas (male-to-male transplants, 189±22%; female-to-male transplants, 197±34%; female-to-female transplants, 93±13%; P=0.0118). Thus, although female aortas transplanted into female recipients remained resistant to AAA, when transplanted into male rats, the observed female resistance was lost.

Male-to-male transplants revealed near-total destruction of the aortic medial elastic lamellar structure, whereas female-to-female transplants had more elastin preservation. Female-to-male transplants followed a similar pattern as male-to-male transplants with near-total destruction of the elastic lamellar structure. Importantly, CD3 staining demonstrated minimal lymphocyte infiltration in any of the transplanted groups. However, ED-1-positive macrophage staining was prominent in the media and adventitia of male-to-male and female-to-male transplanted aortas and less evident in female-to-female transplanted aortas. ED-1-positive macrophages, when quantified, were significantly higher in the male-to-male (68.5±7.4 cells/HPF) and female-to-male transplanted aortas (36.0±1.2 cells/HPF) when compared with female-to-female transplanted aortas (22.4±2.0 positive cells/HPF; P=0.0002). MMP-9 staining was more prominent in male-to-male and female-to-male transplanted aortas transplanted aortas than female-to-female transplanted aortas. Colocalization of ED-1 and MMP-9 demonstrated more costaining in male-to-male transplanted aortas and female-to-male transplanted aortas compared with female-to-female transplanted aortas. MMP-9 mRNA, assessed by quantitative PCR, was also higher in the former aortas (male-to-male transplanted aortas, 0.050±0.002 mRNA copies; female-to-male transplanted aortas, 0.034±0.007 mRNA copies) than in female-to-female transplanted aortas (0.005±0.002 mRNA copies, P=0.0175).

D. Estradiol Effects on Aneurysmal Development

Moderate aortic expansion at 7 days occurred in male rats receiving estradiol and sham control rats being 124%±19% versus 197%±39%, respectively (P=0.010). By 14 days, male rats receiving estradiol had significantly smaller aneurysms (241%±57) compared with sham rats (538%±105, P=0.0226). Elastin fragmentation was less prominent in estradiol treated rats' aortas. ED-1-positive cell counts were 1.8±0.3 cells/HPF in those receiving estradiol versus 5.2±0.5 cells/HPF in sham rats (P=0.0006). Aortic MMP-9 staining was also less evident in the estradiol treated rats compared with the sham rats. Colocalization of aortic ED-1 and MMP-9 demonstrated less prominently stained cells in rats treated with estradiol compared with sham rats. By 7 days after elastase perfusion, estradiol-treated rats exhibited less aortic MMP-9 mRNA expression (0.0017±0.004 mRNA copies) compared with sham rats (0.12±0.04 mRNA copies, P=0.11).

### III. Discussion

As disclosed herein, female rats are partially protected from experimental AAA formation, and male rats consistently form larger AAAs. Female rat aortas subjected to elastase perfusion exhibited less medial wall destruction, fewer infiltrating macrophages, and decreased MMP-9. Furthermore, MMP-9 expression was also decreased in the aortas' of these female rats.

The apparent protection that female aortas exhibited in situ was lost after their transplantation into the male rat, whereas the female aortas transplanted into other female rats maintained their aneurysm resistance. Factors affecting the cardiovascular system, such as increased circulating estrogen known to be present in females, may be potentially associated with the observed AAA resistance. (Mendelsohn M E, Karas R H., N Engl J Med., 340:1801-1811 (1999); Leinwand L A., J Clin Invest., 112:302-307 (2003)).

Numerous studies support a lower incidence of AAA in women compared with men. (Katz D J, et al., J Vasc Surg., 25:561-568 (1997)). In addition, AAAs in women occur nearly a decade later than they do in men, although they are more often juxtarenal compared with infrarenal AAAs. (Velazquez O C, et al., J Vasc Surg., 33(Suppl):84 (2001)). Nonetheless, women have up to 4-times the risk of rupture and death compared with men, (Brown P M, et al., J Vasc Surg., 37:280-284 (2003); Dimick J B, et al., Ann Surg., 235:579-585 (2002)), and have nearly 3-times the complication rate after AAA repair compared with men. (Wolf Y G, et al., J Vasc Surg., 35:882-886 (2002)). Thus, whereas AAAs occur more frequently in men, the clinical sequelae of this disease in women are more disastrous.

One other study has examined both male and female animals in this experimental AAA model. Lee et al., demonstrated no protection from experimental AAA formation in male inducible nitric oxide synthase (iNOS^{-/-}) knockout mice, whereas female iNOS^{-/-} mice had enhanced aortic expansion. (Lee J K, et al., Arterioscler Thromb Vasc Biol., 21:1393-1401 (2001)). Although this study did not evaluate MMPs, it did suggest a gender-related effect of nitric oxide on experimental AAA formation.

The protective role of estrogen and its derivatives during AAA formation receives indirect support from a number of earlier studies. Animals treated with estradiol appear to have increased prostacyclin levels, resulting in improved vasorelaxation (Bolego C, et al., Life Sciences, 60:2291-2302 (1997)) and decreased vascular smooth muscle cell (VSMC) contractility compared with male controls. (Murphy J G, et al., Am J Physiol Cell Physiol., 278:C834-C844 (2000)). In addition, estradiol inhibits medial smooth muscle cell proliferation. (Sullivan T R, Jr., et al., J Clin Invest., 96:2482-2488 (1995)). In an apolipoprotein E-deficient murine model, estradiol was shown to attenuate the development of AAAs. (Martin-McNulty B, et al., Arterioscler Thromb Vasc Biol., 23:1627-1632 (2003)). Furthermore, in postmenopausal women, phytoestrogens result in decreased aortic stiffness. (van der Schouw Y T, et al., Arterioscler Thromb Vasc Biol., 22:1316-1322 (2002)). Thus, estrogen has multiple effects in humans and experimental animals that may be protective of aneurysm development. Embryological aortic development occurs before hormonal variation that occurs during puberty. No apparent differences between male and female native rodent aortas are present histologically. This suggests that estrogens may not act in aortic development, but rather to maintain structure and prevent aortic dilatation. This is supported by the observation that women appear to have a delay in their development of abdominal aortic aneurysms after menopause.

It is generally accepted that macrophages are the primary source for MMP-9 in experimental and human AAAs. As disclosed herein, estradiol inhibited aortic macrophage infiltration and MMP-9 production. Thus, estradiol may effect AAA development by indirectly inhibiting the influx of macrophages and directly by its inhibitory effect on macrophage and smooth muscle cell production of MMPs. Recently, estrogen treatment of U937 cells have been shown to decrease MMP-2 production. Estrogen may effect MMP-9 similarly. It has been shown that estrogen has a direct inhibitory effect on macrophage recruitment, as well as on monocyte chemoattractant protein-1. (Seli E, et al., Fertil Steril., 77:542-547 (2002); Jilma B, et al., Cardiovasc Res., 55:416 (2002); Yamada K, et al., Artery., 22:2435 (1996); Rodriguez E, et al., Life Sciences, 71:2181-2193 (2002)). In a mouse encephalitis model, estrogen inhibited monocyte infiltration into the inflamed tissue. Furthermore, increased estrogen levels in women, including those using estrogen replacement therapy, correlated with reductions in circulating monocyte chemoattractant protein-1 levels.

The primary cell involved in the elastase model is the macrophage, which is the primary source for MMP-9 in human AAAs. (Thompson R W, et al., J Clint Invest., 96:318-326 (1995); Hibbs M S., Matrix Supplement., 1:51-57 (1992)). Other cells that may be involved, such as smooth muscle cells, were consequently not examined in this investigation. In addition, many other proteases known to be upregulated in human AAAs and that are consistently elevated in elastase-perfused experimental AAAs were not examined in the present study. This does not preclude a role for other cell types or other proteases in the observed gender-related differences in experimental AAA formation. Second, transplantation of the aorta, although designed to alter the hormonal environment of the donor aorta, may in and of itself result in a local inflammation in the retroperitoneum. Previous work by Ailawadi et al does suggest that ED-1 cells are increased after transplantation compared with native aortic explants. (Ailawadi G, et al., J Vasc Surg., 37:1059-1066 (2003)). The lack of CD3-positive lymphocytes and the few architectural differences other than those described does suggest that rejection is not involved in this process after transplantation or elastase perfusion. Despite this lack of perceived differences, comparisons between transplanted elastase-perfused aortas and nontransplanted elastase-perfused aortas cannot be made. Third, two different lots of elastase were used in the present investigation and may have resulted in varied results. The first lot was used for elastase perfusion of aortas in the first and second group of experiments (intact and transplanted animals), whereas the latter lot was used for the third group of experiments (those treated with estradiol). These elastase lots were quite different, in as much as the nontransplanted and transplanted elastase-perfused male aortas increased their aortic diameter by 200%, whereas elastase-perfused male aortas used in the estradiol-treatment experiments developed almost 500% increases in their aortic diameter using a different lot of elastase. Such variation has been reported with different lots of elastase despite uniform dose and activity. (Curci J A, Thompson R W., J Vasc Surg., 29:385 (1999)). As a consequence, groups treated with different lots of elastase should not be compared, with comparisons limited only to animals treated with the same lot of elastase.

This investigation supports the theory that gender differences in experimental AAA formation exist that may be related to estrogenic effects on macrophages and MMPs. Gender differences in other cell lines and proteases, as well as cytokines, must be better-evaluated to further completely characterize the disparity between men and women with regard to AAA formation.

### EXAMPLE 2

### Epidemiology of Surgically Treated Abdominal Aortic Aneurysms in the United States, 1988 to 2000

### I. Introduction

Elective abdominal aortic aneurysm (AAA) repair is primarily undertaken to prevent the high mortality associated with rupture. The clinical management of AAAs is very diverse, given differing indications for repair, the procedure's relative complexity, its performance by multiple surgical specialties in a variety of hospitals across the United States, and the wide range of reported outcomes.

The burden to society from AAA disease from data generated in this example is immense in regard to the economics of care and actual lives lost. For example, during a recent decade, the number of elective AAA repairs has averaged 36,000 annually, with an attendant operative mortality near 5%, contributing to 1,800 deaths each year. During the same decade, the number of operations for ruptured AAAs averaged 6,750, with an attendant operative mortality of 46%, resulting in an additional 3,105 deaths each year.

It is generally assumed that the operative mortality associated with AAA rupture represents only 25% of deaths attributed to all AAA niptures, with the remaining 75% succumbing before being treated surgically. If one accepts this assumption, then the deaths occurring before operative therapy can be offered result in an additional 9,315 deaths each year. Thus, the calculated cumulative mortality associated with AAA disease and its treatment carries a loss of 14,220 lives annually. This makes this illness a relatively common cause of death in the United States.

The impetus for the present example was the recognition that little is known about the specific trends in the health care burden attributed to AAAs and whether differences in medical care or variations in the epidemiologic presentation of AAA disease are relevant to medical planning. These data are important as benchmarks when information concerning conventional AAA repair is compared with data for endovascular AAA repair.

The aging population and evolution of the endovascular treatment of AAAs will clearly affect the therapy and outcome of AAA repair during the next decade. (Finlayson S R, et al., J Vasc Surg., 29:973-85 (1999); Sternberg W C III, J Vasc Surg., 36:685-9 (2002); Knickman J R, Health Serv. Res., 37:849-84 (2002); US Census Bureau, Population estimates, Available at: http://eire.census.gov/popest/estimates.php (accessed Feb. 10, 2003)). Furthermore, the availability and more frequent use of noninvasive imaging will increase the recognition of AAAs from the larger reservoir of known, but undiagnosed, AAAs in the general population. Studies on isolated segments of the population, both in the United States and abroad, have resulted in disparate data and inconclusive information regarding therapeutic trends. (Dardik A, et al., J Vasc Surg., 30:985-95 (1999); Hallett J W, et al., J Vasc Surg., 18:684-91 (1993); Heller J A, et al., J Vasc Surg., 32:1091-100 (2000); Hertzer N R, et al., J Vasc Surg., 35:1145-54 (2003); Katz D J, et al., J Vasc Surg., 19:804-17 (1994); Katz D J, et al., J Vasc Surg., 25:561-8 (1997); Lederle F A, et al., N Engl J Med., 346:1437-44 (2002); Pearce W H, et al., J. Vasc Surg., 29:768-76 (1999); Powell J T, et al., Engl J Med., 348:1895-901 (2003); Schermerhorn M L, et al., J Vasc Surg., 31:217-26 (2000)).

In fact, scant national data exists on trends regarding the incidence and outcome of conventional surgery for AAAs. (Dimick J B, et al., Ann Surg., 235:579-85 (2002); Huber T S, et al., J Vasc Surg., 33:304-11 (2001); Lawrence P F, et al., J Vasc Surg., 30:632-40 (1999)). The objective of the present example was to establish a national perspective regarding trends in the surgical treatment of intact and ruptured AAAs.

### II. Methods

A. Data Source

Clinical information was derived from the Nationwide Inpatient Sample (NIS), a 20% stratified random sample of all hospital discharges in the United States. NIS data are maintained by the Agency for Health Care Policy and Research as part of the Healthcare Cost and Utilization Project. All patients who were discharged from 1988 to 2000 with an International Classification of Diseases, Ninth Revision, Clinical Modification (ICD-9-CM) primary procedure code for resection of abdominal aorta with replacement (38.44) or aortobiiliac bypass (39.25) were included in the study. (Public Health Service, Health Care Financing Administration. International classification of diseases, 9th revision, clinical modification (ICD-9-CM). Washington (DC): United States Department of Health and Human Services; (1991)). These procedures were linked to a primary diagnostic code for either AAA (441.4) or ruptured AAA (441.3) to ensure that only patients who underwent operation for AAA disease were included.

B. Outcome Variables

In-hospital mortality was the primary outcome variable studied. Length of stay (LOS) was assessed as a secondary outcome to reflect changes in resource use. Outcomes were segregated according to being associated with intact or ruptured AAA repair. Hospitals were classified into high- and low-volume categories by the median number of AAA operations performed each year (the latter being 31 open AAA repairs/year). True population-based incidence rates of AAA repair were determined by using the hospital sampling weights available from the NIS data set to establish the estimated annual number of procedures performed in the United States. The total number of estimated procedures was then divided by the adult population for each year derived from the US census.

C. Statistical Analysis

Baseline characteristics were compared among patients undergoing intact and ruptured AAA repair. Univariate analyses were performed to assess differences in mortality rates and LOS with linear regression and chi-square tests, and p<0.05 was considered significant. The Statistical Package for the Social Sciences, version 11.0 (SPSS, Chicago, Ill.), was used for all analyses.

### III. Results

A. Patient Characteristics

From 1988 to 2000, 87,728 patients underwent intact AAA repair and 16,295 patients underwent ruptured AAA repair in hospitals included in the NIS data (Table 1). Patients undergoing repair of intact AAAs were different with respect to several characteristics versus those with ruptured AAAs. Of these patients, 85,909 were over the age of 65 years, including 72,270 with intact AAAs and 13,639 with ruptured AAAs. Men outnumbered women 4 to 1 in both the intact and ruptured AAA repair groups.

B. Trends in Incidence

The estimated number of patients with a diagnosis of intact AAA increased from 54.6 to 74.4/100,000 adults from 1988 to 2000 (p<0.001) (Table 2). However, the number of intact AAA repairs remained relatively stable over the 13-year study period, from 18.1 to 16.3 operations/100,000 adults. In contrast, from 1988 to 2000. there has been a decline in the incidence of ruptured AAAs (6.8 to 4.1/100,000; p<0.001), with an accompanying decrease in the incidence of ruptured AAA repair (4.2 to 2.6/100,000; p<0.001).

C. In-Hospital Mortality Rate

The overall in-hospital mortality rate during the 13 years was 4.9% following intact AAA repair and 45.6% following ruptured AAA repair. Among patients older than 65 years, the overall mortality was higher, being 5.5% for intact AAA repair and 48.6% for ruptured AAA repair. Mortality rates changed significantly (p<0.001) for intact AAA repair over the period of study (Table 3), being 6.5% in 1988 and 4.3% in 2000. Similarly, the mortality rate in patients over the age of 65 years decreased from 7.4 to 4.9% (p=0.036) between 1988 and 2000. Among patients under 65 years old, there was also a decrease in operative mortality from 3.3 to 1.4% (p=0.046). The overall mortality following ruptured AAA repair did not change significantly (p=0.225) over the study period (see Table 3). The decline in mortality following intact AAA repair was similar at both high-volume hospitals (greater than 31 repairs/year) and low-volume hospitals (30 or fewer repairs/year) during the study period.

D. Length of Stay

The overall LOS for intact AAA repairs was 9 days (interquartile range [IQR] 7-12 days) and for ruptured AAA repairs was 10 days (IQR 2-18 days). However, the LOS for intact AAA repair decreased significantly (p<0.001) from a median of 11 days in 1988 (IQR 9-15 days) to 7 days in 2000 (IQR 5-10 days). LOS decreases were somewhat less following niptured AAA repair, from a median of 11 days in 1988 (IQR 2-21 days) to 9 days in 2000 (IQR 2-16 days), but this difference remained significant (p<0.001).

### IV. Discussion

The current study provides reliable population-based data on the incidence and outcome of conventional open surgical AAA repair in the United States. These data establish benchmarks regarding the future care of the aging population with AAAs and will allow comparisons with data forthcoming from new endovascular technology for AAA repair. Defined criteria for AAA repair, the risks of untreated AAAs, and specific demographic factors affecting the management of patients with AAAs, have led to better care than in past decades. Thus, it is not unexpected that intact AAA repair in the United States has become increasingly safe during recent years, with lower operative mortality rates and shorter hospital stays. Although repair of ruptured AAAs has not become safer, the frequency of these repairs has decreased. This suggests improved effectiveness in identifying and treating AAAs and possibly a greater patient wellness with treatment of other diseases, such as hypertension, all of which may have lessened the true frequency of aneurysmal rupture.

The decreased frequency of AAA rupture may be attributed to improved measures that identify AAAs before they rupture, but such may not be the case. In fact, although the frequency of diagnosing intact AAAs has increased, there has not been an increase in intact AAA repair. This could be attributed to earlier identification of very small AAAs treated nonoperatively, with more accessible screening processes, such as ultrasonography and computed tomography, as well as an increase in the reservoir of AAAs in the aging population. An alternative perspective is that more effective drug therapies to control hypertension, the reduction in smoking, and the use of statins to reduce low-density lipoprotein cholesterol levels may all lessen oxidative stresses and lower the risk of AAA expansion and rupture.

Decreased LOS for patients undergoing intact AAA repair suggests more efficient postoperative care. The more obvious decline in mortality over time for older patients undergoing AAA repair will become increasingly important with the expected increase in elderly patients requiring care. Understanding past trends when modifying policy to improve future patterns of therapy is essential for accurate health care planning. (Knickman J R, et al., Health Serv Res., 37:849-84 (2002)).

The introduction and increasing use of endovascular treatment of AAAs are likely to affect existing trends regarding traditional open repair of AAAs. Currently, older patients and poor open operative candidates are often served best by endovascular AAA repairs if their anatomy allows endograft placement. In the future, more complex pararenal aneurysms may still require open repair, whereas less complex infrarenal aneurysms will undergo endovascular repair. This may be accompanied by an increase in conventional AAA repair mortality because only the more difficult AAAs would undergo such repairs. Until aortic endovascular technology matures and long-term outcomes following AAA treatment by endografts are better known, it may be difficult to accurately predict the impact of this therapy on open repair.

Many studies have documented the effect of hospital volume on mortality following open AAA repair. These studies uniformly show that high-volume providers have better mortality rates compared with low-volume providers. Surgeon specialty, surgeon volume, and board certification have also been shown to affect mortality rates following AAA repair. Importantly, the present study is the first to document decreased mortality at both highand low-volume hospitals over time in the United States, with better outcomes maintained at highvolume centers. Future regionalization of high-risk patients to high-volume centers may improve the overall outcomes of conventional AAA repair. Concentrating the more complex AAA care in centers of excellence deserves increased attention because the application of endovascular technology is becoming more widespread. This change will have an impact on the training of future vascular surgeons and must be addressed by surgical educators.

Certain limitations of the present example are common to all studies using large administrative databases. Many patient and clinical characteristics are unknown, including aneurysm size, postoperative complications, and long-term outcomes. Nonetheless, its large sample size and representative nature make the NIS review an important device to assess AAA epidemiology on a nationwide basis.

### EXAMPLE 3

### Differential Effect of 17-β-Estradiol on Smooth Muscle Cell and Aortic Explant MMP-2

### I. Introduction

Matrix metalloproteinase-2 (MMP-2) is increased in human abdominal aortic aneurysms (AAAs) and appears critical in the formation of experimental murine AAAs. It is also known that the incidence and size of both human and rat AAAs are greater in males than females. The basis for this gender-related disparity is not known. This Example tested the hypothesis that intrinsic gender-related differences exist in rat aortic smooth muscle cell MMP-2.

### II. Methods

Three sets of experiments comprised this investigation:

Experiment I: Adult male and female rat aortic smooth muscle cells (RASMCs) were grown in Dulbecco's modified Eagle medium (DMEM) with 10% fetal bovine serum (FBS). RASMCs at passages four through eight were stimulated in serum-free media for 48 hours with IL-1β at doses encountered in human aortic aneurysms (2 ng/mL). The culture media was collected, and mRNA was extracted from the RASMCs. After reverse transcription to cDNA, gene expression of MMP-2 and TIMP-2 (a major MMP-2 inhibitor) was measured by real-time polymerase chain reaction. MMP-2 protein levels in conditioned media were measured by Western blotting, with MMP-2 and TIMP-2 activity quantified by standard and reverse gelatin zymography.

Experiment II: Male RASMCs were incubated in DMEM containing 17-β-estradiol and IL-1β, MMP-2 activity in the conditioned media was then determined.

Experiment III: Male rats underwent sustained 17-β-estradiol exposure using extended-release, subcutaneously implanted pellets prior to sacrifice and aortic explanation. The explants were stimulated with IL-1β, and MMP-2 activity in the conditioned media was then determined.

### III. Results

Experiment I: MMP-2 gene expression was 3-fold higher in male compared to female IL-1β stimulated RASMCs (P<0.0001). The MMP-2: TIMP-2 gene expression ratio was greater in male vs. female cells. MMP-2 protein levels (O.D/mg total protein) were 3-fold higher (2.68 vs. 0.96) in male vs. female RASMCs (P=0.003). Gelatinolytic activity (O.D./mg total protein) was more than 6-fold higher (15,010 vs. 2,472) in male vs. female cells (P=0.002).

Experiment II: MMP-2 activity in male RASMCs was not altered by a wide range of 17-β-estradiol concentrations (1×10⁻¹⁰ to 1×10⁻⁶ molar).

Experiment III: Male rats pre-treated with 17-β-estradiol had a 2-fold decrease in MMP-2 activity (O.D./mg protein) in the media of whole-aortic explants (2.0×10⁵ estradiol-treated vs. 4.35×10⁵ control, P=0.002).

### IV. Conclusions

MMP-2 gene expression, protein levels, and gelatinolytic activity were higher in male compared to female RASMCs. 17-β-estradiol did not alter MMP-2 activity in vitro, but in vivo 17-β-estradiol exposure greatly decreased male aortic MMP-2 production. Gender differences in MMP-2 are speculated to be associated with phenotypic differences in human and rat AAA formation.

### EXAMPLE 4

### Gender Differences in Rat Aortic Smooth Muscle Cell Matrix Metalloproteinase-9

### 1. Introduction

Although relatively few studies have investigated the specific contributions of gender to AAA formation, it has been shown that male rats develop experimental aneurysms more frequently than female rats suggesting a possible estrogen-mediated alteration in MMP-9 production. This study was undertaken to investigate fundamental differences in male and female rat aortic smooth muscle cells (RASMCs) with regard to MMP-9 and its natural inhibitor, TIMP-1 (Allaire E, et al., J Clin Inves, 102:1413-1420 (1998); Tilson M D, et al., J Vasc Surg, 18:266270 (1993). Specifically, we hypothesized that the gender differences observed in experimental aneurysm formation are reflected in MMP-9 and TIMP-1 production by RASMCs.

### II. Materials and Methods

A. Cell Culture

Reagents were obtained from Sigma Chemical Co unless otherwise indicated. All experiments were performed with approval of the University of Michigan Committee on Laboratory Animal Medicine. RASMCs were cultured from the abdominal aortas of young (190- to 210-gm) male and female Sprague-Dawley rats (Charles River Laboratories). After animal sacrifice and aortic explanation under general inhalational anesthesia, the aortic tissue was cut into 2-mm² pieces and placed in 60-mm diameter plastic tissue culture dishes. Basement membrane Matrigel (Collaborative Research) was applied to each section of explanted tissue to prevent floating. Cultures were grown in Dulbecco's modified eagle medium (DMEM) containing 10% fetal bovine serum (HyClone Laboratories), 100 U/mL penicillin, 100 μg/mL streptomycin, and glutainine 292 mcg/mL. Tissue culture media and antibiotics were obtained from Gibco. Tissues were incubated at 37° C. in a humidified, 5% CO₂ atmosphere for 4 to 7 days, until spindle-shaped smooth muscle cells were observed extending from the tissue. After removing the explant, the remaining cells were dispersed by treatment with trypsin (Gibco), centrifuged, and resuspended in complete medium, and then placed into 75-cm culture flasks. Post-confluent cultures assumed a hill and valley topography characteristic of SMCs grown in vitro. RASMCs were confirmed by staining with a monoclonal antibody against SMC-specific α-actin.

B. Experimental Interventions

Experimental interventions were carried out with confluent RASMCs at passages 3 through 8 in serum-free DMEM supplemented with antibiotics. Confluent monolayers of RASMCs were incubated in T-75 plates with IL-1β (2 ng/mL, from Sigma) for 72 hours. This concentration of IL-1β is found in human aortic aneurysms. After 72 hours of IL-1β stimulation, the RASMC-conditioned medium was collected for measurements of TIMP-1 activity, MMP-9 activity, and MMP-9 protein levels. The remaining SMCs were lysed in 1% sodium dodecyl sulfate (SDS), and total cellular protein was determined by a bicinchoninic acid protein assay (Pierce). In separate experiments, the previous protocol was followed, but male RASMCs were incubated with 5 different concentrations of 17-β-estradiol (1×10⁻¹⁰ to 1×10⁻⁶ mol/L) for 48 hours with concurrent IL-1β stimulation.

C. Substrate Gel Zymography and Reverse Zymography

Zymography supplies were purchased from Novex. MMP distribution after treatment of RASMC with IL-1β and increasing concentrations of 17-β-estradiol was determined. Gelatin substrate zymograms were prepared using precast 10% SDS-polyacrylamide gels containing 1 mg/mL of gelatin. Equal volumes of experimental media samples were diluted into 2× tris-glycine SDS sample buffer and electrophoretically separated under nonreducing conditions. Proteins were renatured in 2.7% Triton X-100, and the gels were incubated overnight at 37° C. in 50 mmol/L tris-HCl containing 5 mmol/L CaCl₂ and 2% Brij 35. After overnight staining with Coomassie blue R-250 and de-staining for 4 hours with 10% acetic acid and 40% methanol in water, gelatinase activity was evident by clear bands against a dark blue background.

Reverse zymography was performed with RASMC-conditioned media samples after 72-hours IL-1β Pexposure. Gelatin substrate reverse zymograms were prepared using a 15% acrylamide resolving gel containing 1 mg/mL porcine gelatin and conditioned serum-free medium from separate RASMC cultures as a source of progelatinase A. A standard 5% polyacrylamide stacking gel was used. Experimental samples containing equal volumes were diluted into 2× tris-glycine SDS sample buffer and electrophoretically separated under nonreducing conditions. Proteins were renatured in 2 changes of 2.7% Triton X-100 for 60 minutes each. The gels were incubated for 24 hours at 37° C. in 50 mmol/LTris-HCl, 5 mmol/L CaCl₂, and 2% Brij 35. After overnight staining with Coomassie blue R-250 and de-staining for 4 hours with 10% acetic acid and 40% methanol in water, gelatinase inhibitory activity was evident as a blue band against a clear background.

The TIMP-1 band was determined by comparison with authentic TIMP-1 (29 kDa) obtained from Calbiochem. Semiquantitative measurements of TIMP-1 activity were performed by densitometry and corrected to total cellular protein.

D. Western Blot Analysis

Electrophoresis and Western blotting supplies were obtained from BioRad. Equal volumes of media from IL-1β stimulated RASMC cultures were electrophoretically separated on a 7.5% acrylamide gel and blotted onto nitrocellulose membranes. Nonspecific binding was blocked by incubating the membrane overnight in 20 mmol/L tris-HCl (pH 7.5) containing 0.5 M NaCl, 0.1% Tween 20, and 5% nonfat milk. The primary antibody was monoclonal mouse antirat antibody to MMP-9 (NeoMarkers). Peroxidase-coupled goat antimouse antibody was used as a secondary antibody (Calbiochem). Immunoreactive bands were visualized using an electrochemiluminescence detection kit from Amersham, and the amount of protein (corrected to total cellular protein) was measured by densitometry.

E. Semiquantitative Real-Time Polymerase Chain Reaction

Expression of MMP-9 and TIMP-1 messenger RNA (mRNA) was determined using semi-quantitative real-time polymerase chain reaction (RT-PCR). RASMCs treated with IL-1, for 72 hours were lysed, and total cellular RNA was extracted using TRIzol reagent from Life Technologies, and mRNA was purified. Messenger RNA samples were reverse transcribed for 60 minutes at 42° C. using an oligo-(dT) primer and Moloney's murine leukemia virus reverse transcriptase (Life Technologies). Reverse transcription products were used as the substrate for PCR amplification of MMP-9, TIMP-1, and β-actin cDNAs with 5 U/mL Taq DNA polymerase from Promega. Cycling was performed at 94° C. for 2 minutes, followed by 32 cycles of 94° C. for 1 minute, annealing at 57° C. for 1 minute, and extension at 72° C. for 1 minute, and a final incubation at 72° C. for 5 minutes. Amplification was carried out in a GeneAmp 2400 PCR system from Perkin-Elmer. Primers were designed by Primer Premier Software (Premier Biosoft International) and were obtained from Sigma Genosys. The sequences were as follows:

MMP-9 sense 5′-TCT CAA GGA GGT CGG TAT-3′ (SEQ ID NO: 5)

MMP-9 antisense 5′-TCG GGG CAATAA GAA AGG-3′ (SEQ ID NO: 6)

TIMP-1 sense 5′-AAT GCC ACA GGT TTC CGGTTC-3′ (SEQ ID NO: 7)

TIMP-1 antisense 5′-ACA CCC CAC AGC CAG CACTAT-3′ (SEQ ID NO: 8)

β-actin sense 5′-ATG GGT CAG AAG GAT TCC TATGTG-3′ (SEQ ID NO: 9)

β-actin antisense 5′-CTT CAT GAG GTA GTC AGTCAG GTC-3′ (SEQ ID NO: 10)

F. Densitometry

All gel images were acquired using a FOTO/Analyst CCD camera from Fotodyne. Band strength was quantified using GEL-Pro Analyzer software version 3.1 from Media Cybernetics. In cell culture experiments, only pro-MMP-9 bands were observed.

G. Estrogen Pellet Implantation

Male Sprague-Dawley rats (190 g to 210 g) were obtained from Charles River Laboratories. Subcutaneous neck implantation of a 21-day slow release 0.1 mg 17-β-estradiol pellet (Innovative Research of America) was performed under general inhalational anesthesia. This particular estradiol dose results in serum estradiol levels at 2 to 3 weeks of 44.7±6.1 pg/mL compared with 15.0±2.2 pg/mL in control rats (p<0.05). Three weeks after pellet implantation, the infrarenal aorta was explanted under general anesthesia. The aorta was briefly rinsed free of blood and debris with cold 1×PBS containing antibiotics, cut into 1-mm rings, and incubated for 48 hours in 1 mL of serum-free DMEM containing antibiotics and IL-1β. After the incubation period, the conditioned media were collected for zymography as described previously. Tissue pieces were incubated for 24 hours at 37° C. in 500 μL of 1% SDS to extract protein, which was subsequently assayed by bicinchoninic acid protein assay as described previously.

H. Data Analysis

All experiments were performed in triplicate or quadruplicate. An unpaired, two-tailed Student's t-test was used to determine differences in MMP-9 and TIMP-1, with p<0.05 considered significant. Statistical calculations were carried out using GraphPad Prism version 3.0a for Macintosh (GraphPad Software).

### III. Results

A. Gender Differences in MMP-9 from RASMCs

The first set of experiments revealed fundamental differences in MMP-9 gene expression, protein production, and activity in IL-1β-stimulated RASMCs from male and female rats. RT-PCR documented a 10-fold greater relative MMP-9 gene expression in IL-1β-stimulated RASMCs cultured from male aortas than from female aortas (0.14±0.03 versus 0.014±0.007, respectively, p<0.003). After IL-1, stimulation, MMP-9 protein levels in the cell culture media were determined for each gender. Western blot analysis documented greater MMP-9 protein levels in male than in female cell culture media (0.40±0.02 male versus 0.19±0.03 female; optical density [OD]/mg total protein; p<0.005). Baseline IL-1β-stimulated MMP-9 activity as evaluated by gelatin zymography was also significantly greater in male than female RASMC culture media (relative activity 23,320±3,117 male versus 13,680±1,527 female, OD/mg total protein; p<0.01).

B. Tissue Inhibitor of Metalloproteinase-1

RT-PCR documented that gene expression of tissue inhibitor of metalloproteinase-1 was significantly greater in male versus female stimulated RASMCs (3.97±0.44 versus 1.13±0.09; p<0.001). Differences in gene expression correlated with changes in reverse zymography, where TIMP-1 activity (OD/mg protein) was greater in the media from male versus female RASMCs (2.73±0.15 versus 2.02±0.28; p<0.04).

C. Treatment with Estradiol

After characterizing a baseline variation in stimulated MMP-9 and TIMP-1 across gender, the next set of experiments attempted to alter MMP-9 levels by way of pharmacologic hormonal manipulation. In separate cell culture experiments, RASMCs from young male rats were grown in the presence of 17-β-estradiol at doses ranging from 10⁻¹⁰ to 10⁻⁶ mol/L, which included physiologic female levels of estradiol for both rats and humans. Again, the media were subjected to zymographic MMP activity analysis. Despite treatment over a wide range of estradiol concentrations, no difference in MN-9 activity was observed.

After observing that MMP-9 from isolated RASMC culture was not modulated by exogenous estradiol, a third set of experiments was undertaken to evaluate the effect of in vivo delivery of estrogen on MMP-9 activity in aortic whole-tissue explants: male rats were treated for 3 weeks with 17-β-estradiol (0.1 mg, 21-day extended release mg pellet) before aortic explanation and IL-1β stimulation. Conditioned media from estradiol pretreated male rat aortic explants had significantly decreased MMP-9 activity compared with control male aortas (29.9 estrogen-treated versus 75.3 control; OD×10³/mg protein; p<0.03).

### IV. Discussion

This investigation documented intrinsic, gender-related differences in MMP-9 from RASMCs. These findings are consistent with the increased incidence of AAA in men, and with the fact that aneurysms occur more readily in male experimental models of AAA. In addition, this investigation revealed that TIMP-1 gene expression and activity were greater in male than in female RASMCs. Much attention has been directed to the relation between MMPs and TIMPs, and to the inverse relationship observed in human AAAs. The current findings that both MMP-9 and TIMP-1 are greater in males than females do not necessarily contradict earlier observations documenting this inverse relationship because the present comparisons were across genders at baseline; we did not examine the relationship between MMP-9 and TIMP-1 within a single-gender RASMC culture.

A large body of evidence collectively implicates MMP-9 in aneurysm formation. Human MMP-9 gene expression (McMillan W D, et al., Arterioscler Thromb Vasc Biol, 15:1139-1144 (1995); Tamarina N A, et al., Surgery, 122:264-271; discussion 271-262 (1997); Mao D, et al., Ann Vasc Surg, 13:236-237 (1999); Elmore J R, et al., Ann Vasc Surg, 12:221-228 (19998)), protein production, and gelatinolytic activity are higher in aneurysms than in control aortas, and moderately sized AAAs have higher MMP-9 gene expression than do smaller aneurysms or control aortas. Additionally, circulating plasma levels of MMP-9 are higher in patients with AAAs than in those without AAAs, and patients with multiple aneurysms have higher MMP-9 levels than those with solitary aneurysms (McMillan W D, et al., J Vasc Surg, 29:122-127; discussion 127-129 (1999)). In addition, circulating plasma MMP-9 levels have been shown to decrease after AAA repair (Hovsepian D M, et al., J Vasc Interv Radiol, 11:1345-1352 (2000)).

Given the repeated association of MMP-9 and AAAs, multiple investigations have attempted to alter MMP-9 levels in an effort to modify the susceptibility to both experimental and human AAA. Pyo and colleagues (Pyo R, et al., J Clin Invest, 105:1641-1649 (2000)) demonstrated that mice with MMP-9 gene deletions do not form experimental aneurysms, although bone marrow transplantation from wild-type mice restores the aneurysm-susceptible phenotype. Experimental, pharmacologic alterations in MMPs have also been undertaken. Doxycycline, an inhibitor of MMP-9, decreased human plasma MMP-9 levels in vivo over the short-(Thompson R W, et al., Ann NY Acad Sci, 878:159-178 (1999)) and long-term (Baxter B T, et al., J Vasc Surg, 36:1-12 (2002)). Additionally, even at doses relevant to humans, doxycycline inhibited experimental AAA formation in various rodent models (Petrinec D, et al., J Vasc Surg, 23:336-346 (1996); Curci J A, et al., J Vasc Surg, 28:1082-1093 (1998); Prall A K, et al., J Vasc Surg, 35:923-929 (2002); Manning M W, et al., Arterioscler Thromb Vasc Biol, 23:483-488 (2003)).

Despite the large body of evidence linking MMP-9 to the pathogenesis of AAAs, it is well recognized that the cause of AAA is multifactorial (Ailawadi G, et al., J Vasc Surg, 38:584-588 (2003)). In recent basic science reviews, major categories of mechanisms relevant to the formation of AAA have been proposed, including proteolytic degradation of aortic wall connective tissue, inflammation and immune responses, biomechanical wall stress, and molecular-related genetic defects (Ailawadi G, et al., J Vasc Surg, 38:584-588 (2003); Wassef M, et al., J Vasc Surg, 34:730-738 (2001)). Even in these comprehensive reviews, gender differences are not specifically addressed. Gender-related differences in MMPs raise the possibility that hormonal manipulations may be able to modify the risk of aneurysm formation. This has been supported in recent studies in a rodent model in which gender was seen to affect the incidence and extent of experimental AAA formation (Ailawadi G, et al., Arterioscler Thromb Vasc Biol, 24:2116-2122 (2004)).

In this investigation, TIMP-1 gene expression and activity were greater in male than in female RASMCs. The reason for this is unknown. Interestingly, the gene for TIMP-1 is located on the X chromosome, and, generally speaking, inactivation of one of the two female alleles results in equivalent gene expression between genders. If some of this gene inactivation is lost, one would expect to see increased TIMP-1 gene expression in female cells—the opposite of what was observed in this investigation. Indeed, it has been suggested that loss of methylation-mediated gene silencing over time may account for the increased susceptibility of women to some diseases with age. Although beyond the scope of this study, it is possible that supranormal suppression of the female X chromosomes, by chromosomal methylation or other post-transcriptional or posttranslational methods, might account for the varied gene expression and protein product activity reported in these experiments.

The in vitro nature of this investigation is both valuable and limiting. It is important to recognize that in isolated cell culture, male and female RASMCs are fundamentally different with regard to MMP-9 production and activity. Although treatment of male cells with estradiol was not sufficient to alter MMP-9 activity in vitro, MMP-9 activity in the rat aortic explant was altered when estrogen was predelivered to the living animal. Other evidence suggests that the intact (endothelium-containing) aorta acts differently than isolated smooth muscle cell culture. Yen and Lau (Yen C H, Lau Y T, Clin Sci (Lond), 106:541-546 (2004)) performed contractility studies in rat aortic rings from animals pretreated with estradiol and reported decreased contractility compared with rings from untreated rats. This effect was lost in endothelium-denuded rings. The decreased contractility was associated with increased nitric oxide (NO) production, but again, this was only observed in endothelium-intact rings. They concluded that the endothelium is a major source of NO, which subsequently affects smooth muscle cell contractility. Additionally, Gurjar and colleagues (Gurjar M V, et al., J Appl Physiol, 91:1380-1386 (2001)) showed that an NO donor inhibits MMP-9 production in RASMCs, and previous research demonstrated that NO inhibition increased MMP-9 production in both RASMCs30,31 and whole aortic segments (Eagleton M J, et al., J Surg Res, 104:15-21 (2002)). Taken together, this evidence suggests a mechanism for decreased MMP-9 production by aortic tissue segments in this study; estradiol pretreatment to male rats augments endothelial-derived NO, which, in turn, decreases MMP-9 production by the smooth muscle cells.

In addition to the potential impact of the endothelium on RASMC MMP-9 production, the local aortic environment also affects the in vivo aorta. A local factor thought critical in AAA formation is the circulating monocyte and tissue-infiltrating macrophage. Ailawadi (Ailawadi G, et al., Arterioscler Thromb Vasc Biol, 24:2116-2122 (2004)) showed a gender-specific impact of local environment, in which the susceptibility to experimental AAA formation in a rodent model could be altered by aortic transplantation across gender, a finding that corresponded to increased macrophage infiltration in the male recipient.

What is claimed:

1. A method of inhibiting abdominal aortic aneurysm (AAA) formation comprising administering a therapeutically effective amount of a compound selected from the group consisting of estrogen, an estrogen derivative, and an estrogen receptor agonist, to an organism in need thereof.

2. A method of preventing AAA formation comprising administering a therapeutically effective amount of a compound selected from the group consisting of estrogen, an estrogen derivative, and an estrogen receptor agonist, to an organism in need thereof.

3. A method of inhibiting aortic macrophage infiltration comprising administering a therapeutically effective amount of a compound selected from the group consisting of estrogen, an estrogen derivative, and an estrogen receptor agonist, to an organism in need thereof.

4. A method of inhibiting macrophage-derived matrix metalloproteinase (MMP) production comprising administering a therapeutically effective amount of a compound selected from the group consisting of estrogen, an estrogen derivative, and an estrogen receptor agonist, to an organism in need thereof.

5. The method according to claim 4 wherein the matrix metalloproteinase is selected from the group consisting of MMP-1, MMP-2, MMP-3, MMP-9, MMP-12.

6. The method according to claim 5 wherein the matrix metalloproteinase is MMP-9.

7. A method of improving the healing of an AAA repair surgery in an organism, comprising administering a therapeutically effective amount of a compound selected from the group consisting of estrogen, an estrogen derivative, and an estrogen receptor agonist, to an organism in need thereof.

8. A method of reducing the size of one or more AAAs comprising administering a therapeutically effective amount of a compound selected from the group consisting of estrogen, an estrogen derivative, and an estrogen receptor agonist, to an organism in need thereof.

9. The method according to any one of claims 1 through 8 wherein the derivative is estradiol.

10. The method according to any one of claims 1 through 8 wherein the organism in need thereof is a mammal.

11. The method according to claim 10 wherein the mammal is a human.

12. The method according to claim 11 wherein the human is an adult.
